(19) United States
(12) Patent Application Publication
(10) Pub. No.: US 2022/0099650 A1
(43) Pub. Date: Mar. 31, 2022

(54) EARLY WARNING METHOD FOR VADOSE ZONE AND GROUNDWATER POLLUTION IN CONTAMINATED SITE

(71) Applicant: Chinese Research Academy of Environmental Sciences, Beijing (CN)

(72) Inventors: Juan Li, Beijing (CN); Beidou Xi, Beijing (CN); Yang Yang, Beijing (CN); Jun Tang, Beijing (CN); Yang Wang, Beijing (CN); Shunguo Bai, Beijing (CN); Junxiang Shi, Beijing (CN); Lei Chen, Beijing (CN)

(21) Appl. No.: 17/039,073

(22) Filed: Sep. 30, 2020

(51) Int. Cl. G01N 33/18; G01N 27/06; G08B 21/08

(52) U.S. Cl. CPC G01N 33/1886; G08B 21/08; G01N 27/06

(57) ABSTRACT

The present invention belongs to the technical field of warning for petroleum pollution, and particularly provides an early warning method for vadose zone and groundwater pollution in a contaminated site. The present invention can monitor and analyze the conductivity and water content of pore water in a vadose zone in real time and determine the pollution level according to a parameter variation and an analysis on collected samples; and furthermore, the present invention can give multi-level early warnings before groundwater is polluted, accurately determine the pollution level, and provide corresponding measures in time.

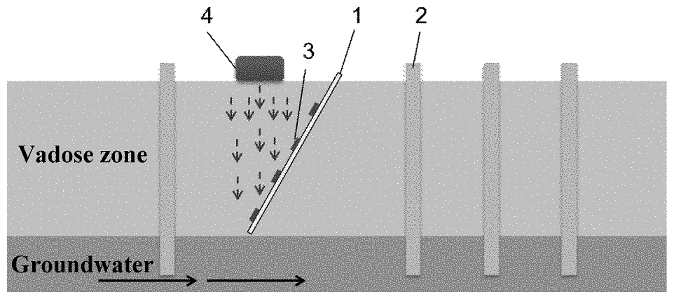

### TECHNICAL FIELD

The present invention relates to the technical field of early warning for petroleum pollution, in particular to an early warning method for vadose zone and groundwater pollution in a contaminated site.

### BACKGROUND

With the development of science and technology and the improvement of industrialization, urbanization has been increasingly accelerated. Some pollutants are prone to permeating into soil and groundwater due to petroleum leakage which may occur during production and transportation of petrochemical enterprises. In this case, environmental pollution occurs. Environmental issues caused by contaminated sites attract increasing attention, and groundwater pollution from the contaminated sites becomes a key environmental issue.

The groundwater pollution is mainly caused by permeation of pollutants in the contaminated sites. The permeation of the pollutants in vadose zones is difficult to monitor and, in most cases, has already caused groundwater pollution when discovered. Once the groundwater is polluted, great harm will be caused to environmental safety and people's life safety, and the groundwater pollution is difficult to treat and usually has high treatment costs due to its invisibility and irreversibility. In view of this, monitoring and early warning are necessary before the groundwater pollution occurs. Therefore, the monitoring on the permeation of the pollutants in the vadose zones and the early warnings before the groundwater is polluted by the pollutants can be performed to take corresponding prevention and control measures. In this way, the pollution is prevented.

A real-time vadose zone monitoring device obliquely penetrates through the vadose zones by means of excavation to monitor whole profiles of the vadose zones. Moreover, the real-time vadose zone monitoring device has environmental friendliness, safety, easy construction, and minimum disturbance to soil layers in the vadose zones, and can monitor physical and chemical indexes of pore water in the vadose zones in real time and collect pore water samples. However, an effective monitoring and warning method for groundwater pollution, which can protect the quality of groundwater below the contaminated sites and other similar buildings by means of the real-time vadose zone monitoring device, has not been put forward.

### SUMMARY

Embodiments of the present invention provide an early warning method for vadose zone and groundwater pollution in a contaminated site, which effectively protects the groundwater quality of petroleum-contaminated sites and other similar buildings and provides a technical reference for early warning for vadose zone and groundwater pollution in the petroleum-contaminated sites.

Embodiments of the present invention provide the following technical solutions:

An early warning method for vadose zone and groundwater pollution in a contaminated site includes the following steps:

(1) surveying hydrogeological conditions of a contaminated site to determine the structure, permeability, and thickness of an aquifer in a vadose zone as well as a buried groundwater depth;

(2) installing a real-time vadose zone monitoring device in a borehole for accommodating the real-time vadose zone monitoring device, where the real-time vadose zone monitoring device includes at least one conductivity monitoring probe for monitoring a real-time variation of the conductivity of pore water in the vadose zone, at least one water content monitoring probe for monitoring a real-time variation of the water content of the pore water in the vadose zone, and at least one device for sampling the pore water in the vadose zone;

(3) reading data from all the monitoring probes every 1-24 h, and giving a first-level early warning when the fluctuation of the conductivity or the water content is greater than ±10%;

(4) after the first-level early warning is given, collecting pore water samples from the vadose zone, and detecting pollutants to determine whether or not the concentration of the pollutants exceeds a concentration limit in a standard;

if the concentration of the pollutants does not exceed the limit, regularly sampling the pore water in the vadose zone to monitor the variation of the concentration of the pollutants; and

if the concentration of the pollutants exceeds the limit, giving a second-level early warning;

(5) after the second-level early warning is given, picking out key pollutants of which the concentration exceeds the concentration limit in the standard to determine a cause of pollution to take prevention and control measures in time for prevention of pollution diffusion, and sampling groundwater in a groundwater monitoring well to detect whether or not the groundwater is polluted by the pollutants;

if the groundwater is not polluted by the pollutants, regularly sampling the pore water in the vadose zone to monitor the migration of the pollutants; and

if the groundwater is polluted by the pollutants, giving a third-level early warning; and

(6) after the third-level early warning is given, sampling the groundwater on a larger scale to determine a pollution range to provide a corresponding treatment scheme.

Preferably, in Step (2), the borehole for accommodating the real-time vadose zone monitoring device obliquely extends to be exactly below the contaminated site and forms an included angle of 30°-60° with the horizontal plane.

Preferably, in Step (2), if the real-time vadose zone monitoring device includes a plurality of conductivity monitoring probes, a plurality of water content monitoring probes, and a plurality of devices for sampling the pore water in the vadose zone, two adjacent conductivity monitoring probes, two adjacent water content monitoring probes, and two adjacent devices for sampling the pore water in the vadose zone are respectively spaced by 0.5-3 m.

Preferably, in Step (3), the first-level early warning is given when the fluctuation of the conductivity or water content of any one of the monitoring probes is greater than ±10%.

Preferably, the standard in Step (4) and Step (5) refers to the “Standard for groundwater quality”.

Preferably, in Step (4), “the concentration of the pollutants does not exceed the limit” particularly refers to that the concentration of any kind of the pollutants in all the pore water samples from the vadose zone does not exceed the concentration limit, and “the concentration of the pollutants exceeds the limit” particularly refers to that the concentration of any kind of the pollutants in any one of the pore water samples from the vadose zone exceeds the limit.

Preferably, in Step (5), “the groundwater is not polluted by the pollutants” refers to that the concentration of the pollutants is not higher than a concentration specified in the standard; and “the groundwater is polluted by the pollutants” refers to that the concentration of the pollutants is higher than the concentration specified in the standard.

Preferably, in both Step (4) and Step (5), the pore water in the vadose zone is sampled every 2-4 months.

The early warning method for vadose zone and groundwater pollution in a contaminated site includes the following steps: (1) surveying the hydrogeological conditions of the contaminated site to determine the structure, permeability, and thickness of the aquifer in the vadose zone as well as the buried groundwater depth; (2) installing the real-time vadose zone monitoring device in the borehole for accommodating the real-time vadose zone monitoring device, where the real-time vadose zone monitoring device includes at least one conductivity monitoring probe for monitoring the real-time variation of the conductivity of the pore water in the vadose zone, at least one water content monitoring probe for monitoring the real-time variation of the water content of the pore water in the vadose zone, and at least one device for sampling the pore water in the vadose zone; (3) reading the data from all the monitoring probes every 1-24 h, and giving the first-level early warning when the fluctuation of the conductivity or the water content is greater than ±10%; (4) after the first-level early warning is given, collecting the pore water samples from the vadose zone, and detecting the pollutants to determine whether or not the concentration of the pollutants exceeds the concentration limit in the standard; if the concentration of the pollutants does not exceed the limit, regularly sampling the pore water in the vadose zone to monitor the variation of the concentration of the pollutants; and if the concentration of the pollutants exceeds the limit, giving the second-level early warning; (5) after the second-level early warning is given, picking out the key pollutants of which the concentration exceeds the concentration limit in the standard to determine the cause of the pollution to take the prevention and control measures in time for the prevention of the pollution diffusion, and sampling the groundwater in the groundwater monitoring well to detect whether or not the groundwater is polluted by the pollutants; if the groundwater is not polluted by the pollutants, regularly sampling the pore water in the vadose zone to monitor the migration of the pollutants; and if the groundwater is polluted by the pollutants, giving the third-level early warning; and (6) after the third-level early warning is given, sampling the groundwater on a larger scale to determine the pollution range to provide the corresponding treatment scheme. The present invention can monitor and analyze the conductivity and water content of the pore water in the vadose zone in real time and determine the pollution level according to a parameter variation and an analysis on the collected samples; and furthermore, the present invention can give multi-level early warnings before the groundwater is polluted, accurately determine the pollution level, and provide corresponding measures in time.

The present summary is provided only by way of example, and not limitation. Other aspects of the present invention will be appreciated in view of the entirety of the present disclosure, including the entire text, claims and accompanying FIGURE.

### BRIEF DESCRIPTION OF DRAWINGS

The sole FIGURE is an installation diagram, showing a warning process for vadose zone and groundwater pollution in a contaminated site, of a device.

While the above-identified FIGURE sets forth an embodiment of the present invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The FIGURE may not be drawn to scale, and applications and embodiments of the present invention may include features, steps and/or components not specifically shown in the drawing.

### DETAILED DESCRIPTION

In the FIGURE, 1. borehole for accommodating a real-time vadose zone monitoring device, 2. groundwater monitoring well, 3. basic unit composed of a conductivity monitoring probe, a water content monitoring probe, and a device for sampling pore water in a vadose zone, 4. pollution source in a contaminated site.

An early warning method for vadose zone and groundwater pollution in a contaminated site includes the following steps:

(1) Survey hydrogeological conditions of a contaminated site to determine the structure, permeability, and thickness of an aquifer in a vadose zone as well as a buried groundwater depth;

(2) Install a real-time vadose zone monitoring device in a borehole for accommodating the real-time vadose zone monitoring device, where the real-time vadose zone monitoring device includes at least one conductivity monitoring probe for monitoring a real-time variation of the conductivity of pore water in the vadose zone, at least one water content monitoring probe for monitoring a real-time variation of the water content of the pore water in the vadose zone, and at least one device for sampling the pore water in the vadose zone;

(3) Read data from all the monitoring probes every 1-24 h, and give a first-level early warning when the fluctuation of the conductivity or the water content is greater than ±10%;

(4) After the first-level early warning is given, collect pore water samples from the vadose zone, and detect pollutants to determine whether or not the concentration of the pollutants exceeds a concentration limit in a standard;

If the concentration of the pollutants does not exceed the limit, regularly sample the pore water in the vadose zone to monitor the variation of the concentration of the pollutants; and

If the concentration of the pollutants exceeds the limit, give a second-level early warning;

(5) After the second-level early warning is given, pick out key pollutants of which the concentration exceeds the concentration limit in the standard to determine a cause of pollution to take prevention and control measures in time for prevention of pollution diffusion, and sample groundwater in a groundwater monitoring well to detect whether or not the groundwater is polluted by the pollutants;

If the groundwater is not polluted by the pollutants, regularly sample the pore water in the vadose zone to monitor the migration of the pollutants; and

If the groundwater is polluted by the pollutants, give a third-level early warning; and

(6) After the third-level early warning is given, sample the groundwater on a larger scale to determine a pollution range to provide a corresponding treatment scheme.

According to the present invention, the hydrogeological conditions of the contaminated site are surveyed to determine the structure, permeability, and thickness of the aquifer in the vadose zone as well as the buried groundwater depth; a survey approach is not specially required, in this case, a conventional survey approach familiar to those skilled in the art can be adopted; and the structure, permeability, and thickness of the aquifer in the vadose zone as well as the buried groundwater depth are determined to determine positions of the real-time monitoring probes and the device for sampling the pore water in the vadose zone in the real-time vadose zone monitoring device.

According to the present invention, preferably, background values of the contaminated site are surveyed to find some regions having excessive pollutants (such as excessive fluorine, arsenic, iron, or manganese in groundwater of the regions) due to their geology, in this way, pollution caused by geology can be excluded. If it is discovered that a certain index in a region exceeds a standard after a survey, when a warning is given according to the technical solution of the present invention, this index is determined based on the background values instead of values specified in the national standard.

According to the present invention, the real-time vadose zone monitoring device is installed in the borehole for accommodating the real-time vadose zone monitoring device and includes at least one conductivity monitoring probe for monitoring the real-time variation of the conductivity of the pore water in the vadose zone, at least one water content monitoring probe for monitoring the real-time variation of the water content of the pore water in the vadose zone, and at least one device for sampling the pore water in the vadose zone.

According to the present invention, in Step (2), preferably, the borehole for accommodating the real-time vadose zone monitoring device obliquely extends to be exactly below the contaminated site and forms an included angle of 30°-60° or even 40°-50° with the horizontal plane; and a method for positioning the borehole for accommodating the real-time vadose zone monitoring device is not specially required, in this case, a conventional technical means familiar to those skilled in the art can be adopted.

According to the present invention, in Step (2), if the real-time vadose zone monitoring device includes a plurality of conductivity monitoring probes, a plurality of water content monitoring probes, and a plurality of devices for sampling the pore water in the vadose zone, two adjacent conductivity monitoring probes, two adjacent water content monitoring probes, and two adjacent devices for sampling the pore water in the vadose zone are preferably respectively spaced by 0.5-3 m or even 1-1.5 m; and the conductivity monitoring probes, the water content monitoring probes, and the devices for sampling the pore water in the vadose zone can be configured at the same position or different positions. In a specific example of the present invention, each basic unit is composed of one conductivity monitoring probe, one water content monitoring probe, and one device for sampling the pore water in the vadose zone; and by means of an array of all basic units, the conductivity monitoring probes, the water content monitoring probes, and the devices for sampling the pore water in the vadose zone are arrayed.

According to the present invention, the data from all the monitoring probes is read every 1-24 h and is read preferably every 12-16 h; and the first-level early warning is given when the fluctuation of the conductivity or the water content is greater than ±10%; and in Step (3), preferably, the first-level early warning is given when the fluctuation of the conductivity or water content of any one of the monitoring probes is greater than ±10%.

According to the present invention, after the first-level early warning is given, the pore water samples are collected from the vadose zone; the pollutants are detected to determine whether or not the concentration of the pollutants exceeds the concentration limit in the standard; if the concentration of the pollutants does not exceed the limit, the pore water in the vadose zone is regularly sampled to monitor the variation of the concentration of the pollutants; and if the concentration of the pollutants exceeds the limit, the second-level early warning is given.

According to the present invention, the standard particularly refers to the “Standard for groundwater quality” or specifically the “Standard for groundwater quality” (GB/T 14848-2017).

According to the present invention, in Step (4), the pore water in the vadose zone is sampled preferably every 2-4 months or even every three months.

According to the present invention, in Step (4), “the concentration of the pollutants does not exceed the limit” particularly refers to that the concentration of any kind of the pollutants in all the pore water samples from the vadose zone does not exceed the concentration limit, and “the concentration of the pollutants exceeds the limit” particularly refers to that the concentration of any kind of the pollutants in any one of the pore water samples from the vadose zone exceeds the limit.

According to the present invention, after the second-level early warning is given, the key pollutants of which the concentration exceeds the concentration limit in the standard are picked out to determine the cause of the pollution to take the prevention and control measures in time for the prevention of the pollution diffusion, and the groundwater in the groundwater monitoring well is sampled to monitor whether or not the groundwater is polluted by the pollutants; If the groundwater is not polluted by the pollutants, the pore water in the vadose zone is regularly sampled to monitor the migration of the pollutants; and

If the groundwater is polluted by the pollutants, the third-level early warning is given.

According to the present invention, the standard particularly refers to the “Standard for groundwater quality” or specifically the “Standard for groundwater quality” (GB/T 14848-2017); and the prevention and control measures are not specially required, in this case, a technical means familiar to those skilled in the art can be adopted.

According to the present invention, “monitor the migration of the pollutants” particularly refers to that the variation of the concentration of the pollutants at each detecting point along with time is monitored, and water samples are compared from top to bottom to find a difference.

According to the present invention, in Step (5), “the groundwater is not polluted by the pollutants” preferably refers to that the concentration of the pollutants is not higher than a concentration specified in the standard and is particularly not higher than the concentration specified in a class III water quality standard from the standard, and “the groundwater is polluted by the pollutants” preferably refers to that the concentration of the pollutants is higher than the concentration specified in the standard and is particularly higher than the concentration specified in the class III water quality standard from the standard.

According to the present invention, in Step (5), the pore water in the vadose zone is sampled preferably every 2-4 months or even every 3 months.

According to the present invention, after the third-level early warning is given, the groundwater is sampled on a larger scale to determine the pollution range to provide the corresponding treatment scheme; and the specific treatment scheme is not specially required, in this case, a technical means familiar to those skilled in the art can be adopted.

The technical solution provided by the present invention will be described in detail in combination with the following examples, but they should not be construed as limiting the claimed scope of the present invention.

### Example 1

A petroleum processing workshop in a petroleum refinery is selected as a monitoring and warning object. The petroleum refinery beside a river is 500 m away from the river. A buried groundwater depth is 1.8-2.0 m; groundwater feeds into the river; and a sandy soil layer below the surface is 0-0.5 m thick, a silt or silty clay layer below the sandy soil layer is about 1.2 m thick, and an aquifer as a gravel layer is 3.5 m thick and has a permeability coefficient of 75.40 m/d. Due to high permeability of the aquifer, pollutants will rapidly migrate along with the groundwater if entering the groundwater, resulting in pollution in a larger range. In view of this, a real-time vadose zone monitoring device is installed below a sewage treatment plant. Key pollutants include petroleum hydrocarbons, benzene, toluene, sulfides, and cyanides.

A well obliquely sunk in a petroleum-contaminated site forms an included angle of 55° with the surface. If a groundwater depth is 2 m, a total length of the well is 2.44 m, and the distance between the top of the well and the bottom of the well is 1.4 m in the horizontal direction. Conductivity monitoring probes as well as water content monitoring probes and devices for sampling pore water in a vadose zone are spaced by 1 m and are arranged at two points on the real-time vadose zone monitoring device to cover the whole profile of the vadose zone.

A conductivity and a water content are recorded every 24 h (every day). If the conductivity or the water content of a day is 10% higher than that of the previous day, a first-level early warning is given.

After the first-level early warning is given, pore water samples are collected from the vadose zone; and if it is detected that the concentration of the benzene is 100 mg/L and is higher than that of 10 mg/L in a class III water quality standard from the “Standard for groundwater quality”, a second-level early warning is given.

After the second-level early warning is given, groundwater in a groundwater monitoring well is sampled; and if it is detected that the concentration of the benzene is 5 mg/L and is lower than that of 10 mg/L in the class III water quality standard from the “Standard for groundwater quality”, the groundwater is sampled every months to detect the concentration of the benzene for prevention of pollution diffusion.

### Example 2

An industrial sewage treatment plant is selected as a monitoring and warning object. A groundwater level of the sewage treatment plant is 13 m, a sandy soil layer below the surface is 0-13 m thick, a loam or clay layer in the middle is about 0.5 m thick, and an aquifer as a coarse sand layer is about 10 m thick and has a permeability coefficient of 105 m/d. Due to high permeability of the aquifer, pollutants will rapidly migrate along with the groundwater if entering the groundwater, resulting in pollution in a larger range. In view of this, a real-time vadose zone monitoring device is installed below the sewage treatment plant. Key pollutants include pollutants with a high chemical oxygen demand (COD), ammonia nitrogen, sulfides, and so on.

A well obliquely sunk close to an aeration tank and a sedimentation tank forms an included angle of 55° with the surface. If a groundwater depth is 13 m, a total length of the well is 16 m, and the distance between the top of the well and the bottom of the well is 9 m in the horizontal direction. Conductivity monitoring probes as well as water content monitoring probes and devices for sampling pore water in a vadose zone are spaced by 3 m and are arranged at five points on the real-time vadose zone monitoring device to cover the whole profile of the vadose zone.

A conductivity and a water content are recorded every 24 h (every day). If the conductivity or the water content of a day is 10% higher than that of the previous day, a first-level early warning is given.

After the first-level early warning is given, pore water samples are collected from the vadose zone; and if it is detected that the key pollutants exist, pore water in the vadose zone is sampled every other quarter to detect the pollutants for prevention of the pollution.

The above descriptions are merely preferred implementations of the present invention. It should be noted that a person of ordinary skill in the art may further make several improvements and modifications without departing from the principle of the present invention, but such improvements and modifications should be deemed as falling within the protection scope of the present invention.

What is claimed is:

1. An early warning method for vadose zone and groundwater pollution in a contaminated site, comprising the following steps:
(1) surveying hydrogeological conditions of a contaminated site to determine a structure, permeability, and thickness of an aquifer in a vadose zone as well as a buried groundwater depth;
(2) installing a real-time vadose zone monitoring device in a borehole for accommodating the real-time vadose zone monitoring device, wherein the real-time vadose zone monitoring device comprises at least one conductivity monitoring probe for monitoring a real-time variation of a conductivity of pore water in the vadose zone, at least one water content monitoring probe for monitoring a real-time variation of a water content of the pore water in the vadose zone, and at least one device for sampling the pore water in the vadose zone;
(3) reading data from all the monitoring probes every 1-24 hours, and giving a first-level early warning when a fluctuation of the conductivity or the water content is greater than ±10%;
(4) after the first-level early warning is given, collecting pore water samples from the vadose zone, and detecting pollutants to determine whether or not a concentration of the pollutants exceeds a concentration limit in a standard;
if the concentration of the pollutants does not exceed the limit, regularly sampling the pore water in the vadose zone to monitor a variation of the concentration of the pollutants; and
if the concentration of the pollutants exceeds the limit, giving a second-level early warning;
(5) after the second-level early warning is given, picking out key pollutants of which a concentration exceeds the concentration limit in the standard to determine a cause of pollution to take prevention and control measures in time for prevention of pollution diffusion, and sampling groundwater in a groundwater monitoring well to detect whether or not the groundwater is polluted by the pollutants;
if the groundwater is not polluted by the pollutants, regularly sampling the pore water in the vadose zone to monitor a migration of the pollutants; and
if the groundwater is polluted by the pollutants, giving a third-level early warning; and
(6) after the third-level early warning is given, sampling the groundwater on a larger scale to determine a pollution range to provide a corresponding treatment scheme.

2. The early warning method according to claim 1, wherein in Step (2), the borehole for accommodating the real-time vadose zone monitoring device obliquely extends to be exactly below the contaminated site and forms an included angle of 30°-60° with a horizontal plane.

3. The early warning method according to claim 2, wherein, in Step (2), the real-time vadose zone monitoring device comprises a plurality of conductivity monitoring probes, a plurality of water content monitoring probes, and a plurality of devices for sampling the pore water in the vadose zone, and wherein two adjacent said conductivity monitoring probes, two adjacent said water content monitoring probes, and two adjacent said devices for sampling the pore water in the vadose zone are respectively spaced by 0.5-3 m.

4. The early warning method according to claim 1, wherein, in Step (2), the real-time vadose zone monitoring device comprises a plurality of conductivity monitoring probes, a plurality of water content monitoring probes, and a plurality of devices for sampling the pore water in the vadose zone, and wherein two adjacent said conductivity monitoring probes, two adjacent said water content monitoring probes, and two adjacent said devices for sampling the pore water in the vadose zone are respectively spaced by 0.5-3 m.

5. The early warning method according to claim 1, wherein in Step (3), the first-level early warning is given when the fluctuation of the conductivity or water content of any one of the monitoring probes is greater than ±10%.

6. The early warning method according to claim 1, wherein the standard in Step (4) and Step (5) is the “Standard for groundwater quality” (GB/T 14848-2017).

7. The early warning method according to claim 6, wherein in Step (4), “the concentration of the pollutants does not exceed the limit” particularly refers to that the concentration of any kind of the pollutants in all the pore water samples from the vadose zone does not exceed the concentration limit; and
“the concentration of the pollutants exceeds the limit” particularly refers to that the concentration of any kind of the pollutants in any one of the pore water samples from the vadose zone exceeds the limit.

8. The early warning method according to claim 6, wherein in Step (5), “the groundwater is not polluted by the pollutants” refers to that the concentration of the pollutants is not higher than a concentration specified in the standard; and
“the groundwater is polluted by the pollutants” refers to that the concentration of the pollutants is higher than the concentration specified in the standard.

9. The early warning method according to claim 6, wherein in both Step (4) and Step (5), the pore water in the vadose zone is sampled every 2-4 months.

10. The early warning method according to claim 1, wherein in Step (4), “the concentration of the pollutants does not exceed the limit” particularly refers to that the concentration of any kind of the pollutants in all the pore water samples from the vadose zone does not exceed the concentration limit; and
“the concentration of the pollutants exceeds the limit” particularly refers to that the concentration of any kind of the pollutants in any one of the pore water samples from the vadose zone exceeds the limit.

11. The early warning method according to claim 1, wherein in Step (5), “the groundwater is not polluted by the pollutants” refers to that the concentration of the pollutants is not higher than a concentration specified in the standard; and
“the groundwater is polluted by the pollutants” refers to that the concentration of the pollutants is higher than the concentration specified in the standard.

12. The early warning method according to claim 1, wherein in both Step (4) and Step (5), the pore water in the vadose zone is sampled every 2-4 months.
